(19) United States
(12) Patent Application Publication
(10) Pub. No.: US 2013/0280354 A1
(43) Pub. Date: Oct. 24, 2013

(54) Composition and Method for Using Sage

(71) Applicants: Raza Bashir, Oakville (CA); Craig Wissent, Oakville (CA); Jason Peters, Oakville (CA)

(72) Inventors: Raza Bashir, Oakville (CA); Craig Wissent, Oakville (CA); Jason Peters, Oakville (CA)

(73) Assignee: NORTHERN INNOVATIONS HOLDING CORP, Oakville (CA)

(21) Appl. No.: 13/454,288

(22) Filed: Apr. 24, 2012

(51) Int. Cl. A61K 36/537; A61P 21/00; A61K 36/82

(52) U.S. Cl. 424/729; 424/746

(57) ABSTRACT

A composition containing Salvia officinalis for improving a person's response to thermogenesis is provided. The composition may be used in methods of administration of Salvia officinalis before exercise. The composition may also be administered with thermogenic substances before exercise or before consuming a meal to improve a person's response to thermogenesis.

### FIELD OF THE INVENTION

This invention relates generally to nutritional supplements, and, in particular, to a novel method and composition beneficial to a subject's energy, cognitive focus, comfort, and perceived exertion during exercise and subsequent recovery after exercise or in conjunction with the administration of a thermogenic substance.

### BACKGROUND OF THE INVENTION

Physical exercise that occurs during athletic events, such as various sports competitions, and routine training for such, and regular recreational exercise, causes varying levels of stress to the body. The physical work done during exercise is associated with force exertion (both actual and perceived), temperature changes, increases in sweat excretion, and increases in uptake of oxygen, water and other key metabolites used to replenish energy stores.

The use of dietary supplements among recreational and elite athletes has become increasingly popular due to studies suggesting improvements in performance. For some dietary supplements, such as vitamins and minerals, administration time is not necessarily governed by timing of exercise. However, other dietary supplements specifically formulated to provide specific benefits for exercise, such as pre-workout, intra-workout, and post-workout supplements, or thermogenic supplements for aiding in weight loss or also to provide benefits for exercise, are taken at specific times in relation to an exercise session or prior to a meal in order to achieve specific potential benefits. These benefits may be numerous and include ergonomic improvement of exercise through increased endurance, energy through stimulant effects or thermogenic effects, and improved recovery during brief periods of rest or low activity, or improved recovery after completion of an exercise session or athletic event. Most pre-workout supplements are focused only on providing increased energy for exercise. Thermogenic substances may also be used to help control body weight, either for maintaining a current body weight, or for reducing body weight, particularly fat, outside the context of exercise.

While some of the effects of exercise an individual experiences during exercise or in response to a thermogenic supplement are tolerable or, in some cases desirable, these vary among individuals in terms of the effect's presence and the degree of the effect. There is a need for a dietary supplement that improves additional aspects of a subject's exercise session or response to thermogenic supplements.

### SUMMARY OF THE INVENTION

Embodiments of the invention provide compositions for administration before exercise, or in conjunction with a thermogenic supplement before exercise or before a meal. The compositions comprise Salvia officinalis, which has the beneficial effect of tempering or reducing some of the effects commonly experienced during or after exercise or in response to a thermogenic substance, such as excessive sweating and increased temperature, thereby improving subjective feelings of comfort and cognitive focus, without reducing the beneficial effects of thermogenesis, whether the thermogenesis is due to the thermogenic effect of exercise, food consumption, or thermogenic substances.

In one embodiment, a method of improving exercise capacity of a human subject is provided by administering to the subject a composition comprising Salvia officinalis or an extract thereof, prior to commencement of the exercise.

In another embodiment, a method of improving the response of a human subject to a thermogenic substance is provided by including in a composition that includes the thermogenic substance, Salvia officinalis or an extract thereof.

In another embodiment, a composition comprising Salvia officinalis or an extract thereof and a thermogenic substance is provided. The thermogenic substance is one of caffeine or sources of caffeine, yohimbine or sources of yohimbine, synephrine or sources of synerphrine, Green tea or Green tea extracts, forskolin or sources of forskolin, L-carnitine or salts of L-carnitine, and hydroxycitric acid or sources of hydroxycitric acid.

### DETAILED DESCRIPTION OF THE EMBODIMENTS

### Definitions

A used herein, the terms ‘composition’ or ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract or derivative of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, Soft-Gel™ caplets, gel-caps and the like, not associated with food.

As used herein, the term “derivative” refers to a compound, such as a salt, ester, or amine, which can readily supply a closely related biologically active compound, either upon administration or upon exposure to specific environment al conditions, such as pH, temperature, etc. For example, a “derivative” of caffeine can be a salt, ester or amine of caffeine, so long as the derivative can readily supply biologically active caffeine. For a given compound, the skilled person in the art will readily recognize and envisage those compounds which should be considered “derivatives.”

Thus, for example, a derivative of a particular substance may comprise a form of that substance which has been modified through reaction. Other derivatives are forms of a given substance that are precursors of that substance which would give rise to that substance after modification. Reactions involved in the formation of derivatives include, but are not limited to: hydroxylation, esterification, amide formation and salt formation.

As used herein, the term “thermogenesis” is understood to mean the process of heat production in or by the body, particularly increased heat production by or in the body. It is also understood that thermogenesis may be increased by a number of mechanisms including, but not limited to diet-induced thermogenesis, exercise-induced thermogenesis, and non-exercise-associated thermogenesis. As used herein, a “thermogenic substance/supplement” is understood to mean any substance, which when ingested by a subject increases thermogenesis. This is understood to be associated with any or all of increased heat production by the body, increased energy expenditure, and increased metabolic rate.

Sage (Salvia officinalis) has long been used as a culinary seasoning in food and also as an herbal medicinal product, most typically in form of an infusion (tea). Traditional uses include treatment of inflammation, fever, indigestion, and excessive perspiration. It has also been used externally for wound treatment, as an astringent, and an antifungal and antibacterial. The leaves are the most common part used and principal components include thujone, 1,8-cineole, camphor, rosmarinic acid, ursolic acid, and carnosic acid.

More recent research suggests some efficacy for Sage in reducing perspiration and inflammation. There is also evidence that Sage provides some cognitive and mood benefits, particularly in terms of Alzheimer's disease, the mechanism implicated being cholinesterase inhibiting properties for Sage. Overall, Sage, in various forms, has been associated with antioxidant activity, anti-cholinesterase activity, anti-inflammatory activity, anti-lipase activity, and various behavioral effects such as improved memory.

Sage, in accordance with embodiments of the present invention, may be in any form or derivative, as is known in the art. For example, according to one embodiment, Sage may be provided as whole, fresh or dried, Salvia officinalis plant, or any part thereof, such as leaves, flowers, stems, or roots. In preferred aspects, the plant part used is the leaf. In preferred aspects, the leaves of Salvia officinalis plants are used. Additional aspects employ an extract of Salvia officinalis plant, or any part thereof. Such extracts are preferably standardized to at least one principal component. In the form of dried/powdered leaves, the composition may contain, per serving, from about 50 mg to about 1 g of Sage. More preferably, the amount of Sage per serving is from about 10 mg to 500 mg. Most preferably, the composition may contain, per serving, about 150 mg of Sage leaf powder.

According to another embodiment of the present invention, Salvia officinalis may be provided as an extract of Salvia officinalis plant, or any part thereof, aqueous or alcoholic extracts. Extraction techniques suitable for use with plants and plant material are well known in the art. It is herein understood that the goal of extraction is to concentrate or separate soluble plant metabolites, including desired active components, from structural plant matter, or otherwise unwanted plant matter. The final form of an extract may be liquid (aqueous or oil), semi-solid, or powdered. The Salvia officinalis extract may be standardized to one or more specific components of the extract. Basic extracts may contain a complex mixture of many plant metabolites, such as alkaloids, glycosides, terpenoids, flavonoids and lignans. The most common form of extract used to administer Sage is as a hot water infusion, or extract of dry leaves, i.e. a tea. Other suitable forms of extracts include alcoholic extracts such as ethanolic or methanolic. Additionally suitable extraction methods are hydroalcoholic extracts using solvent mixtures of alcohol and water of various portions. Distillation may also be used to extract essential oil.

In some embodiments, the Salvia officinalis is provided as one component of a multi-component composition for administration before exercise. Therefore, the compositions, in all embodiments, contain at least two Components—Salvia officinalis being one of the two. Many ingredients are known and commonly used for pre-workout supplementation. Such ingredients include, but are not limited to: carbohydrates or sources of carbohydrates such as glucose, dextrose, maltodextrin, and waxy maize; anti-oxidants or sources of anti-oxidants such as plants or plant extracts such as Acai, pomegranate, green tea, or grape; various amino acids or amino acid-related ingredients such as L-leucine, L-valine, L-isoleucine, 4-Hydroxyisoleucine, L-arginine, L-phenylalanine, L-citrulline, L-carnitine, L-ornithine, beta-alanine, taurine, L-glutamine, creatine and various form/salts, vinpocetine; and other plants or plant extracts purported to provide ergogenic benefits such as Rhodiola Rosea, Huperzia serrata (supplying huperzine), ginseng (Panax ginseng, Panax quinquefolius, Panax notoginseng and Panax japonicus), and Yohimbe (supplying yohimbine). Alternatively, active components found in such plants may be provided in purified form or in synthetic form.

Caffeine is a popular ingredient alone or as part of pre-workout supplements to provide energy and cognitive focus during exercise and its benefit is primarily due to its action as a central nervous system stimulant but may also spare glycogen by promoting the utilization of fat for energy. In a preferred embodiment, Salvia officinalis is provided in a composition with caffeine or a source of caffeine in a composition for administration before exercise. The preferred form of caffeine is caffeine anhydrous; however, the source of caffeine may be other known sources such as Coffea Arabica, Coffea canephora, Camellia sinensis, Ilex paraguariensis, Paullinia cupana, Theobroma cacao, and kola nut, or derivatives as is known in the art, provided that the desired amount of caffeine is provided. The amount of caffeine per serving of the composition is from about 100 to 375 mg of caffeine. More preferably, the amount of caffeine per serving of the composition is from about 200 to 300 mg. Most preferably, the amount of caffeine per serving of the composition is about 270 mg.

In addition to the foregoing, compositions of the present invention include formulations further comprising additional active ingredients and/or inactive ingredients, including solvents, diluents, suspension aids, thickening or emulsifying agents, sweeteners, flavorings, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions, which may also be suitable for use in formulations of the present invention. Except insofar as any conventional carrier medium is incompatible with the ingredients of the invention, such as by producing any undesirable effect or otherwise interacting in a deleterious manner with any other ingredient(s) of the formulation, its use is contemplated to be within the scope of this invention.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a capsule or as a powdered beverage mix.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

### Example 1

The following ingredients when combined with excipients (magnesium stearate and silica) are mixed and packed into hard gelatin capsules at a concentration of two capsules per serving to be taken with water about 30 minutes before exercise.
Caffeine Anhydrous 0.27 g, Coffea canephora extract, bean 0.2 g, Sage powder (Salvia officinalis), leaf 0.15 g, L-Theanine 0.1 g, Yohimbe Bark extract powder (8-9.6% yohimbine) 0.056 g, Yerba mate extract (Ilex paraguariensis), leaf 0.05 g, and Rhodiola Rosea extract 0.05 g.

### Example 2

The following ingredients when combined with excipients (microcrystalline cellulose, magnesium stearate, and silica) are mixed and packed into hard gelatin capsules at a concentration of three capsules per serving to be taken with water about 30 minutes before exercise.
Caffeine Anhydrous 0.1 g, Sage powder (Salvia officinalis), leaf 0.1 g, Cocoa extract (Theobroma cacao), seed—std. (6% theobromine) 0.03 g, Yohimbe Bark extract Powder (8-9.6% yohimbine) 0.038 g.

### Example 3

The following ingredients when combined with excipients (magnesium stearate and silica) are mixed and packed into hard gelatin capsules at a concentration of two capsules per serving to be taken with water about 30 to 60 minutes before exercise.
Caffeine Anhydrous 0.27 g, Coffee canephora extract, bean 0.2 g, Sage powder (Salvia officinalis), leaf 0.15 g, L-Theanine 0.1 g, and Rhodiola Rosea extract 0.05 g.

### Example 4

The following ingredients when combined with excipients (magnesium stearate and silica) are mixed and packed into hard gelatin capsules at a concentration of two capsules per serving to be taken with water about 30 minutes before exercise.
Caffeine Anhydrous 0.3 g and Sage powder (Salvia officinalis), leaf 0.2 g.

The scope of the claims should not be limited by the preferred embodiments or the embodiments set forth in the examples, but should be given the broadest interpretation consistent with the description as a whole. Those skilled in the art will recognize and appreciate where modifications may be made.

All publications which are cited herein are hereby specifically incorporated by reference into the disclosure for the teachings for which they are cited.

What is claimed is:

1. A method of improving exercise capacity of a human subject, the method comprising administering to the subject a composition comprising Salvia officinalis or an extract thereof, prior to commencement of the exercise.

2. The method of claim 1, wherein the composition is administered from about 15 minutes to 60 minutes prior to the commencement of the exercise.

3. The method of claim 2, wherein the composition further comprises a thermogenic substance.

4. The method of claim 3, wherein the thermogenic substance is caffeine.

5. The method of claim 4, wherein the caffeine is caffeine anhydrous or is from at least one of Coffee Arabica, Coffee canephora, Camellia sinensis, Ilex paraguariensis, Paullinia cupana, Theobroma cacao, or kola nut.

6. A method of improving the response of a human subject to a thermogenic substance, wherein the thermogenic substance is a component of a composition, the method comprising administering to the subject the composition that contains the thermogenic substance which composition also comprises Salvia officinalis or an extract thereof.

7. The method of claim 6, wherein the composition is administered prior to the commencement of exercise.

8. The method of claim 7, wherein the composition is administered from about 15 minutes to about 60 minutes prior the commencement of exercise.

9. The method of claim 8, wherein the thermogenic substance is caffeine.

10. The method of claim 9, wherein the caffeine is caffeine anhydrous or is from at least one of Coffea Arabica, Coffea canephora, Camellia sinensis, Ilex paraguariensis, Paullinia cupana, Theobroma cacao, or kola nut.

11. The method of claim 6, wherein the composition is administered prior to the consumption of a meal.

12. The method of claim 11, wherein the composition is administered from about 15 minutes to about 60 minutes prior the consumption of a meal.

13. The method of claim 12, wherein the thermogenic substance is caffeine.

14. The method of claim 13, wherein the caffeine is caffeine anhydrous or is from at least one of Coffea Arabica, Coffea canephora, Camellia sinensis, Ilex paraguariensis, Paullinia cupana, Theobroma cacao, or kola nut.

15. The method claim 3, wherein the improving exercise comprises improving at least one parameter of exercise capacity of the subject, the at least one parameter of exercise capacity is at least one of energy, cognitive focus, comfort, perceived exertion, or recovery of skeletal muscle contractility.

16. The method of claim 15, wherein the composition is administered from about 15 minutes to 60 minutes prior to the commencement of the exercise.

17. The method of claim 16, wherein the thermogenic substance is caffeine.

18. The method of claim 17, wherein the caffeine is caffeine anhydrous or is from at least one of Coffea Arabica, Coffea canephora, Camellia sinensis, Ilex paraguariensis, Paullinia cupana, Theobroma cacao, or kola nut.

19. A composition comprising Salvia officinalis or an extract thereof and a thermogenic substance, wherein the thermogenic substance is one of caffeine or sources of caffeine, yohimbine or sources of yohimbine, synephrine or sources of synerphrine, Green tea or Green tea extracts, forskolin or sources of forskolin, L-carnitine or salts of L-carnitine, and hydroxycitric acid or sources of hydroxycitric acid.
